(19) United States
(12) Patent Application Publication
(10) Pub. No.: US 2007/0233221 A1
(43) Pub. Date: Oct. 4, 2007

(54) Esophageal dilation and stent delivery system and method of use

(75) Inventor: Gottumukkala S. Raju, League City, TX (US)

Correspondence Address: LOCKE LIDDELL & SAPP LLP;ATTN: IP DOCKETING, 600 TRAVIS, 3400 CHASE TOWER, HOUSTON, TX, 77002-3095, US

(73) Assignee: The Board of Regents, the University of Texas System

(21) Appl. No.: 11/395,727

(22) Filed: Mar. 31, 2006

(51) Int. Cl. A61F 2/06

(52) U.S. Cl. 623/1.11

(57) ABSTRACT

Methods and assemblies for dilating and/or delivering a stent into a body cavity or vessel is described. The assembly is particularly suited for the dilation, delivery and fixation of a stent in a body cavity, such as in a stricture in the esophagus, and includes a guidewire, an endoscope having a dilator cap affixed to its distal end, a stent, and a flexible stent delivery device. The dilator cap can be customized to various sizes, depending upon the interior diameter of the stricture to be dilated, and can be interchanged with the other dilator caps on the distal end of the endoscope. The endoscope-dilator assembly can be slid distally down the guidewire to the stricture to be dilated, and the stent can then be delivered distally along the guidewire using a delivery device. The stent delivery device and the endoscope-dilator assembly can be retracted proximally along the guidewire through the interior of the stent, leaving the stent in place in the stricture.

### FIELD OF THE INVENTION

This disclosure relates generally to dilation of strictures and a body implantable treatment device, and more particularly to a dilation and stent delivery system and other prostheses intended for fixation in body lumens, including the esophagus, as well as methods of use thereof.

### DESCRIPTION OF RELATED ART

An esophageal stricture is a gradual narrowing of the esophagus, which can lead to swallowing difficulties. The strictures can be caused by scar tissue that builds up in the esophagus. When the lining of the esophagus is damaged, scarring develops, and the lining of the esophagus becomes stiff. In time, as this scar tissue continues to build up, the esophagus begins to narrow in that area, resulting in swallowing difficulties (dysphagia). One of the conditions that can lead to esophageal strictures is gastroesophageal reflux disease, wherein excessive acid is refluxed from the stomach up and into the esophagus. This causes an inflammation in the lower part of the esophagus. Scarring typically results after repeated inflammatory injury and healing, reinjury and rehealing. This scarring will produce damaged tissue in the form of a ring that narrows the opening of the esophagus.

Benign esophageal strictures are also a relatively common complication in esophageal diseases, including cancer. In fact, the incidence of esophageal cancer has risen in recent decades accounting for 1.5% of all invasive cancers [Urso, J. A., et al., St. Francis Journal of Medicine, 1996]. Occurring anywhere in the esophagus, the cancer can exist as a stricture, mass, or plaque, and often results in painful dysphagia (difficulty swallowing and the sensation that food is sticking on the way down the esophagus).

Typical treatments of dysphagia include both surgical and endoscopic intervention to insert a stent or similar prosthesis which serves to bridge the obstruction in the esophagus and reestablish luminal patency, as well as treatments with pharmaceuticals such as proton pump inhibitors and corticosteroids which can keep certain inflammatory-related strictures from reforming.

While surgical removal or treatment of strictures, especially carcinomas in the esophagus, can sometimes be effective, the majority of patients exhibiting esophageal tumors are not candidates for surgery. Repeated dilation (stretching) of the esophagus is typically the only option, and can provide only temporary relief. Such difficult or refractory cases were often treated by laser therapy with an Nd:YAG laser [Fishman, V., et al., Gastrointest. Endosc., 53(1): pp. 128-130 (2001)], or by intubation using rigid plastic prostheses. Such techniques, while effective, also have notable disadvantages.

For example, photodynamic laser therapy is expensive, typically requiring several treatment sessions before results are evident. Additionally, tumor recurrence is frequent, on the order of 30-40 percent [Moghissi, K., et al., Tecnol. Cancer Res. Treat., 2(4): pp. 319-326 (2003)]. Further, certain submucosal tumors, and certain pulmonary tumors causing dysphagia by esophageal compression, cannot be treated by laser therapy techniques. Rigid plastic prosthesis insertion is also not without problems. These rigid plastic stents are typically large, having diameters in the range of 12 mm or more, and often have outer end flanges to aid in holding them in place. Placement of such rigid, non-elastic plastic stents can be traumatic due to their large size, and can often result in perforation of the esophageal wall. Additionally, even with the end flanges, these prostheses can be the subject of migration and late pressure necrosis [Ferguson, D. D., Dis. Esophagus, 18(6): pp. 359-364 (2005)].

However, while dilation of esophageal strictures is a useful approach to the alleviation of the symptoms typically associated with this complication, such as dysphagia, it is not without associated risks and complications. Key among these complications are the processes of dilating the stricture and implanting a treatment device such as a stent. Current practices involve the dilation of the stricture using a wire guide and a catheter over the guide to insert a dilator within the stricture. The dilator typically contains a stent that is then expanded using a balloon [Therasse, E., et al., Radiographics, 23: pp. 89-105 (2003)]. The problem with this technique is that it requires the use of a long dilator (on the order of 90 mm or more), and the expandable stents used are subject to recoil, necessitating several procedures to insert different, larger stents so as to effectively open the stricture to a point that dysphagia is alleviated.

The problem with stent recoil has been attempted to be addressed by the use of metal stents, such as surgical stainless steel stents. In this procedure, a guide wire and X-ray monitoring are used to guide the metal stent in a compressed state over the guide wire, down the esophagus, and into the stricture. Pulling a thread or inflating a balloon, allowing the stent to expand to a preset diameter, then releases the metal stent and the guide wire is retracted. However, using this technique, no dilator is used, and so the width of the stent is constrained, typically to about 4-8 mm. In order to widen the stricture to a point that relieves at least some of the dysphagia associated with it, several procedures must typically be performed in order to gradually widen the gap in the stricture by inserting consecutively larger stents, a process that can be both costly and painful. Additionally, metal stents can become incorporated into the wall of the esophagus by means of epithelial growth around the stent struts. Resultant and subsequent sludge formation and/or ingrowth or overgrowth of the stent may cause obstruction of the metal endoprosthesis, necessitating surgical removal and re-stenting.

Plastic stents, such as those plastic prostheses made of Teflon®, polyethylene, and polyurethane, have also been used in the dilation of esophageal strictures, but with less success than that shown with metal stents. In a typical procedure, a two-stage insertion process is followed, wherein an endoscope is inserted, and a guide-wire is then inserted and positioned using the endoscope. The endoscope is removed, and using X-ray techniques to follow the placement and insertion of the stent, a catheter (13-14 mm in diameter) that contains the plastic stent is placed in the stricture, simultaneously dilating the stricture. The stent is then released from the catheter, and the catheter is removed, allowing the plastic stent to remain behind and hold open the stricture. However, the use of these stents typically result in incrustation and sludge (obstruction) formation of varying degrees, which can eventually lead to premature occlusion of the stented stricture. Additionally, these stents have been shown to be less cost-effective and have higher reintervention costs than other dilation prostheses [Lammer, J., et al., Radiology, 201: pp. 167-172 (1996)].

Therefore, there remains a need for a dilation and stent delivery system and method for dilating and/or delivering a stent to a body vessel or cavity that can improve over the prior limitations and problems.

### SUMMARY

In at least one embodiment, the disclosure provides an assembly for the delivery of a prosthesis in a body vessel or cavity of a mammal, the assembly comprising: a guide wire; an endoscope having a distal end and a proximal end; a dilator cap having a distal end, a proximal end, and a tapered shape wherein the proximal end has a diameter greater than the diameter of the distal end; wherein the proximal end of the dilator cap is coupled to the distal end of the endoscope. In accordance with this embodiment, the assembly can further comprise a flexible stent delivery device as well as a stent removably coupled to the device, and the dilator cap can be transparent or translucent. Further, the dilator cap of this embodiment can be selected from a kit comprising a plurality of dilator caps, the plurality of which can each have a different proximal end diameter.

In another embodiment, the disclosure further provides a method for delivering a prosthesis into a body vessel or cavity of a mammal using assemblies disclosed herein, wherein the method comprises inserting a guide wire within a body vessel or cavity, coupling a first dilator cap to the distal end of an endoscope to form a endoscope-dilator assembly, extending the endoscope-dilator assembly through the interior of the vessel or cavity to a predetermined point so as to effectively dilate the predetermined point, and withdrawing the endoscope-dilator assembly. In accordance with certain aspects of this embodiment, this method further comprises selecting a second dilator cap from a kit comprising a plurality of dilator caps, the second dilator cap having proximal end diameter different from the proximal end diameter of the first dilator cap; coupling the second dilator cap to the distal end of the endoscope to form a second endoscope-dilator assembly; and, extending the second endoscope-dilator assembly through the interior of the vessel or cavity to the predetermined point.

In another embodiment, the present disclosure provides an assembly for the delivery and fixation of a stent in a body vessel or cavity, the assembly comprising a guide wire; an endoscope having a distal end and a proximal end; a dilator cap having a distal end, a proximal end, and a length no more than one quarter of the length of the endoscope; a stent; and a flexible stent delivery device, the dilator cap being substantially transparent or translucent and coupled to the distal end of the endoscope. In accordance with this embodiment, the dilator cap can be selected from a kit comprising a plurality of dilator caps, each of which have different proximal end and/or distal end diameters. Additionally, the stent can further comprise one or more apertures or rings circumferentially located on a flange of the stent.

In a further embodiment, the disclosure further provides a method for delivering a prosthesis or stent into a body vessel or cavity, comprising: providing a stent delivery assembly; inserting a guide wire having a distal tip and a proximal end within a body vessel or cavity until the guide wire distal tip reaches a desired location in the body vessel or cavity; extending an endoscope having a dilator cap attached to the distal end thereof through the interior of the vessel or cavity to a predetermined point; inserting a stent through the vessel or cavity to the predetermined point with a stent delivery device; withdrawing the endoscope-dilator assembly from the body vessel or cavity; and withdrawing the stent delivery device from the body vessel or cavity, wherein the method is performed independent of an expanding device, such as a balloon expander. In accordance with this embodiment, the method can further comprise attaching the stent to a wall of the vessel or cavity using an attachment means, such as a surgical suture, that is capable of passing through one or more apertures or rings in a flange of the stent and into a wall of the vessel or cavity.

The disclosure further provides a method for delivering a stent into the esophagus of a mammal for treating esophageal strictures, comprising: extending a guide wire into the esophagus and extending the guide wire past a stricture in the esophagus; inserting a endoscope having a dilator cap coupled thereto (an endoscope-dilator assembly) into the esophagus, extending through the esophageal stricture; inserting a stent into the esophagus, over the endoscope; inserting the stent into the esophageal stricture using a pusher catheter; and withdrawing the endoscope having the dilator cap affixed thereto from the stricture, while simultaneously maintaining the stent in position with the pusher catheter. This method can be performed independent of an expanding device associated with the dilator or the stent. In further accordance with this embodiment of the present disclosure, the pusher catheter, guide wire, and endoscope-dilator cap assembly can be withdrawn separately, or in combination, or simultaneously. Additionally, the method can further comprise attaching the stent to a wall of the vessel or cavity using an attachment means that is capable of passing through one or more apertures or rings in a flange of the stent and into a wall of the vessel or cavity.

In yet another embodiment, the present disclosure provides a kit for use in endoscopic surgery and similar surgical procedures. Such a kit comprises a plurality of conically-shaped dilator caps having a distal end, a proximal end, and a tapered edge between the distal end and the proximal end. These dilator caps are translucent or transparent, have a proximal end with a diameter ranging from about 5 mm to about 15 mm, and have a distal end with a diameter ranging from about 2 mm to about 8 mm. In accordance with this embodiment, one or more of the dilator caps in the kit can comprise at least one radio-opaque element. Further, such dilator caps can optionally have one or more graduations on or within a surface.

In a further embodiment, the current disclosure provides a stent for use in therapeutic applications, the stent comprising a distal flange, a proximal flange, a medial sleeve portion intermediate between the distal flange and the proximal flange, and a support structure within the medial sleeve, wherein the proximal flange comprises one or more apertures or rings for the attachment of the stent to a biological body using any number of attachment means.

### DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 illustrates a side elevational view of an exemplary dilation and stent delivery system.

FIG. 2 illustrates a longitudinal side view of a portion “A” of the endoscope-dilator assembly and guide wire forming part of the exemplary delivery system.

FIG. 3 illustrates a distal end view of the endoscope and the guide wire of FIG. 2, taken along line B-B.

FIGS. 4A-4F illustrate the general method of dilating and delivering a stent into a body cavity using a delivery assembly.

FIG. 5 illustrates a fragmentary and partial side section view of the assembly in a vessel.

FIG. 6A illustrates a detailed view of an endoscope-dilator assembly.

FIG. 6B illustrates a detailed view of an alternate embodiment of the endoscope-dilator assembly.

FIG. 7 illustrates a plurality of dilator caps adopted to be used with a delivery system.

FIG. 8A illustrates a cut-away view of an assembly in accordance with the present disclosure, during placement in a human patient.

FIG. 8B illustrates a top view of the assembly of FIG. 8A, taken along line C-C, looking downward from the proximal end of pusher 100 towards the distal end of guide wire 14.

While the concepts disclosed herein are susceptible to various modifications and alternative forms, only a few specific embodiments have been shown by way of example in the drawings and are described in detail below. The figures and detailed descriptions of these specific embodiments are not intended to limit the breadth or scope of the concepts or the appended claims in any manner. Rather, the figures and detailed written descriptions are provided to illustrate the concepts to a person of ordinary skill in the art and to enable such person to make and use the concepts.

### DETAILED DESCRIPTION OF THE INVENTION

One or more illustrative embodiments are presented below. Not all features of an actual implementation are described or shown in this application for the sake of clarity. It is understood that in the development of an actual embodiment incorporating the present invention, numerous implementation-specific decisions must be made to achieve the developer's goals, such as compliance with system-related, business-related, government-related and other constraints, which vary by implementation and from time to time. While a developer's efforts might be complex and time-consuming, such efforts would be, nevertheless, a routine undertaking for those of ordinary skill the art having benefit of this disclosure.

In general terms, the applicant has created an assembly and method for dilating and introducing a stent or similar prosthesis into a vessel or cavity, such as through a stricture within the esophagus of a mammal, which is easier and quicker than previous dilation methods. The assembly is customizable to the diameter of dilation of the specific patient, thereby minimizing the need for repeat dilations in order to obtain the desired degree of dilation. Additionally, the assembly and associated methods described herein can allow for a reduced number of times that a stricture is required to be re-dilated, due to a decreased incidence of collapse or stent failure.

Strictures suitable for treatment with the stent delivery system and associated methods of the present invention include but are not limited to carcinomas, inflammatory is scarring, tissue scarring, and inflamed tissue. Similarly, body vessels or cavities wherein the delivery system and associated methods can be applied include but are not limited to the throat, esophagus, intestinal tract, trachea, and colon.

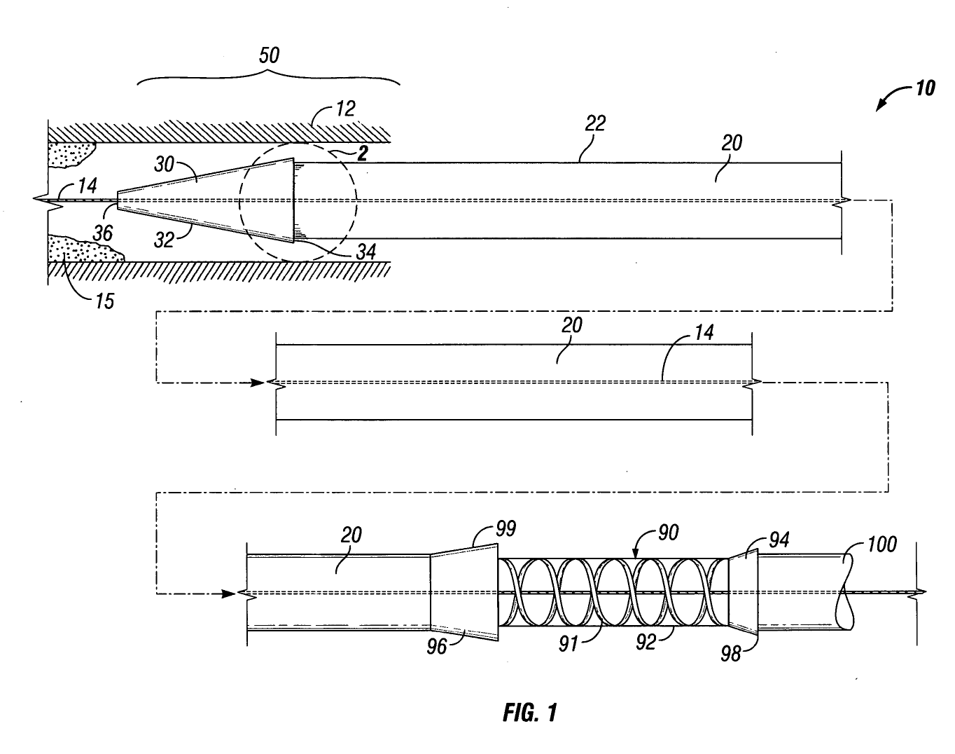

Referring now to the figures, FIG. 1 is an illustration of a side elevational view of an exemplary dilation and stent delivery system in accordance with the present disclosure. The dilation and stent delivery assembly 10 for use in a vessel or cavity circumscribed by a wall 12 having a stricture 15 is illustrated broadly in FIG. 1. Such an assembly includes a guide wire 14, an endoscope 20, a dilator cap 30, a stent 90, and a pusher catheter 100. As will be described in more detail below, endoscope 20 and dilator cap 30 are coupled together such that they form a endoscope-dilator assembly 50. Stent 90, described in more detail below, is in at least one embodiment a non-expandable, non-rigid stent comprising a plurality of support structures 91, such that the use of a balloon expander or similar device with the assembly and methods of the present invention is unnecessary.

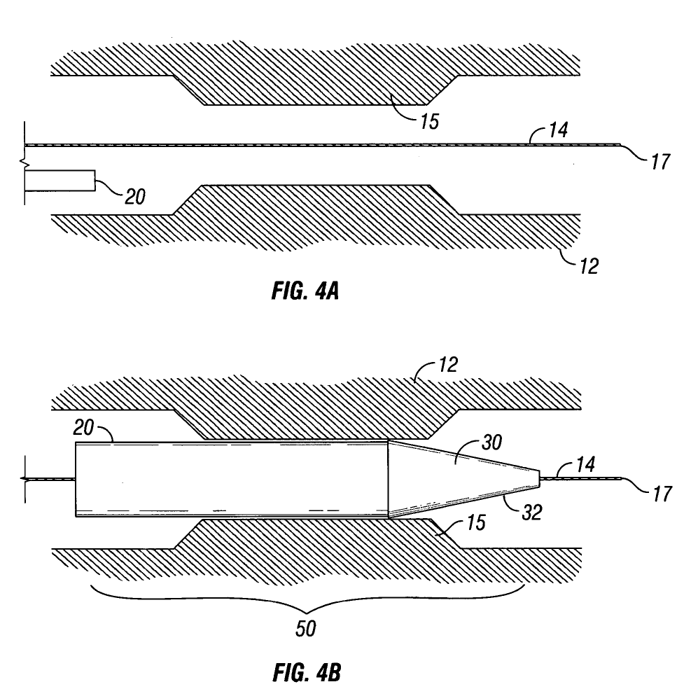

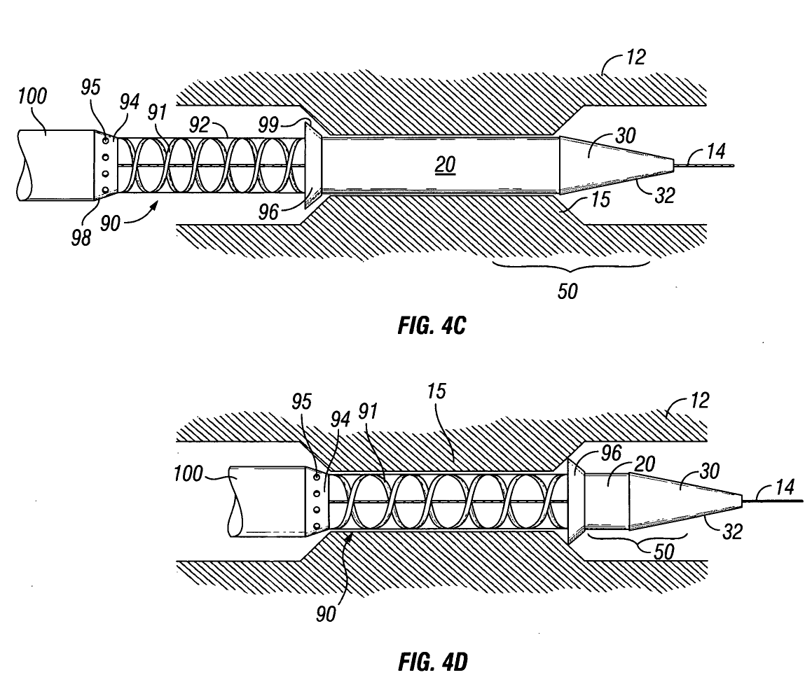

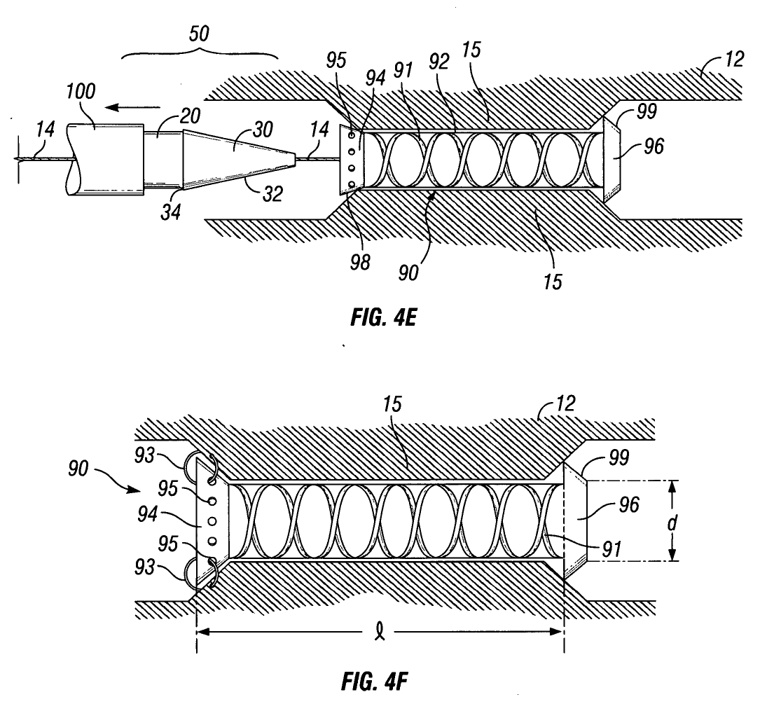

Guide wire 14 as shown in FIG. 1 can be any suitable guide wire, such as an Amplatz extra stiff (AES) guide wire (0.035 inch diameter). Typically, guide wire 14 can have a diameter from about 0.1 mm to about 2 mm. Guide wire 14 can further comprise a guide wire tip at its distal end 17 (see FIG. 4A) for use in threading guide wire 14 so that the guide wire can guide the assembly and stent along the desired vessel or cavity for proper positioning of the endoscope-dilator assembly 50 and the stent 90. The guide wire 14 can pass through the inner bore of the dilator cap 30, and the inner bore of the endoscope 20, as will be described in more detail in reference to FIG. 3.

Returning to FIG. 1, the dilator cap 30 comprises a substantially conical shaft having a tapered edge 32 extending along a longitudinal axis and having a distal end 36 and a proximal end 34. The dilator cap 30 is generally tapered to allow the dilator to follow the guide wire 14 through the confined portions of a stricture 15 in a vessel or cavity (such as the esophagus) 12 more gently. Dilator caps suitable for use in the present invention can be fabricated from a flexible, elastomeric material such as an EVA (ethylene vinyl acetate) copolymer, or from a stiff material such as 90/10 high-density polypropylene. Additionally, and as discussed in more detail below, dilator cap 30 can include one or more components, such as barium sulfate, to make it radiopaque. In accordance with one aspect of the present invention, the dilator cap 30 can comprise a portion that is transparent or opaque, so that the user can look through the dilator at the mucosa of the stricture or vessel using the endoscope, eliminating the need for a long dilator.

The dilator cap 30 can range in diameter at its proximal end 36 from about 2 mm to about 20 mm, advantageously from about 6 mm to about 16 mm, and ranges in length from about 2 cm to about 20 cm, and advantageously from about 2 cm to about 6 cm in length, making it a “dilator cap”. Additionally, as indicated above, the dilator cap is advantageously substantially transparent in nature, or made of a suitable translucent material so as to allow viewing therethrough with the endoscope. In this manner, different size dilator caps can offer a plurality of different dilations of a stricture, depending upon the individual need. Generally, the dilator cap 30 affixed to the distal end of endoscope 20 has an overall length no more than one quarter of the length of the endoscope being used.

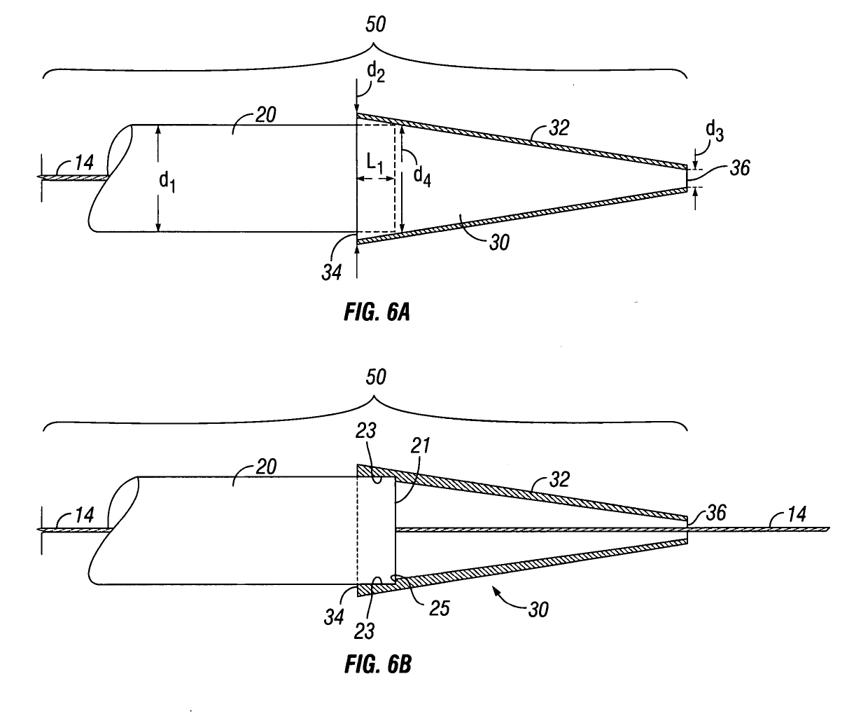

Endoscope 20 includes an elongated, cylindrical shaft having a longitudinally extending central bore, a distal end 21, and a proximal end (not shown) connected to viewing assemblies know in the art. In accordance with the present invention, the combination of dilator cap 30 with endoscope 20 comprises an endoscope-dilator assembly 50. As illustrated therein, dilator cap 30 is coupled to the distal end of endoscope 20, such that the proximal end 34 of dilator cap 30 is proximate to the distal end of the endoscope. Assembly 50 can be brought together by either the use of an appropriate adhesive, by use of similarly sized openings that can be pressed or otherwise is held together, or other methods of coupling distinct pieces together, as discussed in more detail below in reference to FIG. 6A and FIG. 6B.

In a typical example, the user selects a dilator cap 30 having a desired diameter opening at the proximal end 34 and at the distal end 36. The distal end of an endoscope 20 is pushed into the interior of dilator cap 30, from the proximal end 34 towards the distal end 36, so that the dilator cap 30 forms an exterior sheath over a portion of the distal end of endoscope 20. If the diameters are appropriately sized, the endoscope can be held in place relative to the dilator cap 30. In certain aspects of the present disclosure, the endoscope can fit into a preformed bore within the dilator cap 30. Alternatively, the relative outside diameter of the endoscope and the inner diameter of the dilator cap 30 along the conical shape can simply be appropriately sized to allow for the coupling of the pieces together.

It will be apparent to those of skill in the art that this sequence can be varied in a number of acceptable ways. For example, instead of inserting endoscope 20 through the proximal opening of dilator cap 30, dilator cap 30 can be pulled over the exterior of endoscope 20, thereby forming assembly 50. In one aspect of the present invention, the dilator can be held in place by the use of a suitable adhesive, such as a surgically-acceptable glue. For example, this could be an appropriate manner of forming an endoscope-dilator assembly 50 if the opening diameter of the dilator cap 30 was substantially equivalent to the outer diameter of the endoscope to be used.

Stent or prosthesis 90, as illustrated generally in FIG. 1, will now be described in more detail, although other features and aspects of stent 90 will also be discussed in reference to FIGS. 4C-4F, below. The prostheses or stent 90 illustrated in FIG. 1 (as well as other figures herein), can be constructed to address various concerns which arise from the use of solid plastic stents or expandable stents. As shown in the figures, stent 90 can be of mesh or open weave construction, comprised of multiple braided and helically wound strands or support structures 91. Stent 90 has a medial sleeve or region 92, a proximal flange 94 and distal flange 96. The proximal and distal flanges of the stent can be tapered radially outward, as indicated at edge 98, and tapered radially inward as indicated at edge 99. Such radially flaring flanges are included to improve stent fixation adjacent the stricture, once the stent has been placed. Additionally, in the instance that the esophagus of a patient requires re-dilation at some point in the future, proximal flange 94 allows for the easy removal of the stent using forceps or similar tools.

When stent 90 is in its relaxed or normal configuration as shown in the figures, medial region 92 can have a diameter ranging from about 10 to about 20 mm, and the flanges can have a diameter ranging from about 20 mm to about 30 mm. In at least one embodiment, the support structures 91 forming the stent 90 can be formed of body compatible metal such as stainless steel or nitinol and can be about 0.50 mm or less in diameter. As seen in FIG. 1 (and also in FIGS. 4C, 4D and 4E, below), the axial length of the medial region 92 is on the order of about one-third or more of the axial length of the stent 90. For example, and referring to FIG. 4F, the stent structure 90 can have a longitudinal length (l) ranging from about 1.0 mm to about 15 cm, and an outer diameter (d) ranging from about 7 mm to about 20 mm, the width depending upon the stricture to be treated, and the size of the endoscope-dilator assembly to be used.

Returning to FIG. 1, prostheses or stents 90 suitable for use and placement within a vessel or cavity can include those stents having flexible support structures 91 embedded in a hollow, cylindrical casing of a synthetic, elastomeric material. The support structure of such stents is typically composed of two or more shaped filaments (e.g., zig-zag shaped) which extend parallel to the longitudinal axis of the casing of the stent. Examples of such suitable stents include, but are not limited to, the Ultraflex stent (Microvasive/Boston Scientific, Natick, Mass.), the Wallstent (Schneider, Inc.), the Z-stent (Microvasive/Boston Scientific, Natick, Mass.), the Wilson-Cook stent (Wilson-Cook Medical, Winston-Salem, N.C.), the Key-Med stent (Atkinson), the Buess stent, and the is Polyflex® stent (Boston Scientific, Natick, Mass.), as well as numerous other silicone-covered self-expanding metal stents. Suitable stents also can include those stents comprising nickel-titanium, non-magnetic Ni—Ti alloys such as nitinol, ELGILOY® (Elgiloy Specialty Metals, Elgin, Ill.), nickel-rich nitinol, memory metals, stainless steel, plastics, and gold as flexible support structures, all of which may be imparted with resilience to urge outward while maintaining non-deforming, radial flexibility. Other stent materials and designs are possible given the disclosure contained herein, including expandable stents.

While flanges 94 and 96 can be open, medial region 92 is advantageously circumscribed, i.e. completely covered, with a continuous polymeric film, such as silicone. The implantable medical device has been described using a structure such as a stent adapted for introduction into a vessel or cavity of a patient. The stent structure 90 described above can also be composed of a continuous, covering film having at least one surface suitable for contact with the wall of the vessel or cavity, or a stricture present therein. A number of useful coatings, such as a degradable mucin or other coating to minimize friction, as well as heparin, fibrin, or fibrin-elastic compounds can be used. Such an outer film can be included so as to help facilitate passage of food and fluids through the interior of the stent, while the outer coating of film can act to help resist hyperplasia, and exert a constant, gentle pressure against the stricture, thus helping to adapt to normal esophageal peristalsis while maintaining luminal patency. In the instance of the silicone coating, the silicone film can be applied by dip coating of stent 90, in which event the film initially covers one of the flanges, and is removed from that flange prior to using the stent. The thickness of the film can measure from about 0.0001″ to about 0.0004″ (0.1-0.4 mil, or 0.0025-0.01 mm), advantageously having a thickness of about 0.0002 or 0.0003″ (0.2-0.3 mil, or 0.0050-0.0075 mm) to about 0.1 inch (2.54 mm). One exemplary thickness of the silicone film can be in the range of about 0.003 inches to about 0.01 inches (about 0.075 mm to about 0.25 mm), and is controlled primarily by the number of dip coating applications. More specifically, from three to six dip coatings can result in the thicknesses above.

Another aspect of the stents 90 suitable for use with the present invention is radiopacity. Radiopacity permits the physician to visualize the procedure involving the stent through use of fluoroscopes or similar radiological equipment, without the use of X-ray equipment. Such radiopacity can be added to the stents through coating processes such as sputtering, plating, or co-drawing gold or similar heavy metals onto the stent. Radiopacity can also be included by alloy addition. One specific approach can be to alloy the nickel-titanium of the stent supports with a ternary element.

In at least one embodiment, the stent 90 used with the system of the present invention can be a radiopaque stent that is constructed from a tubular-shaped body having a thin wall defining a strut pattern, wherein the tubular body includes a superelastic, nickel-titanium alloy, and the alloy further includes a ternary element to provide radiopacity, the ternary element being selected from the group of chemical elements consisting of iridium, platinum, gold, rhenium, tungsten, palladium, rhodium, tantalum, silver, ruthenium, or hafnium. In accordance with this aspect, the stent can also have a radiopaque core.

The stent structure 90 can also be treated so as to be radioactive, in order to minimize hyperplasia and excessive neointimal growth. In a further embodiment, the stent structure 90 can further comprise an outer bioactive material layer that can be adhered to the outer base surface of the covering film using any number of techniques known to those of skill in the art. The bioactive material layer comprises one or more therapeutic compounds or combinations thereof, in a therapeutically effective amount. Suitable therapeutic compounds include anticancer agents, NSAIDs, corticosteroids, local anesthetic agents, and antibiotics, as well as combinations thereof. A “therapeutically effective amount” of such agents, as used herein, is meant to be a nontoxic but sufficient amount of an active therapeutic agent to provide the desired therapeutic effect. Such a therapeutically effective amount can be an amount ranging from about 0.5 μm/day to about 50 g/day. Therapeutically effective amounts of anticancer agents, as described herein, refers to an amount of one or more anticancer agents sufficient to inhibit tumor proliferation when administered to a mammal in need of curing, alleviation, or prevention of tumors, especially a human suffering from proliferation of tumor cells, in order to inhibit the growth of the tumor cells.

Anticancer agents suitable for use in association with the stents useful in the present invention include taxoids (such as paclitaxel (Taxol®) or docetaxel (Taxotere®), alkylating agents (such as cyclophosphamide, isosfamide, melphalan, hexamethyl-melamine, thiotepa or dacarbazine), topoisomerase inhibitors (such as camptothecin and its derivatives including CPT-11, topotecan and pyridobenzoindole derivatives), antimetabolites (such as pyrimidine analogues, for instance 5-fluarouracil and cytarabine, or its analogues such as 2-fluorodeoxycytidine, or folic acid analogues such as methotrexate, idatrexate or trimetrexate), vinca alkaloids (such as vinblastine or vincristine or their synthetic analogues such as navelbine), epidophylloptoxins (such as etoposide or teniposide), antibiotics (such as such as daunorubicine, doxorubicin, bleomycin or mitomycin), enzymes such as L-asparaginase, and various agents such as estramustine, procarbazine, mitoxantrone, platinum coordination complexes such as cisplatin or carboplatin, and biological response modifiers or growth factor inhibitors such as interferons or interleukins, as well as combinations thereof.

Nonsteroidal anti-inflammatory drugs (NSAIDs) suitable for use as therapeutic agents in association with the stent system of the present invention include propionic acid derivatives such as ketoprofen, ibuprofen, flurbiprofen, naproxen, and the like; piperazines and piperazine derivatives; spiro-hydantoin derivatives; indoles and indole derivatives; imidazoles and imidazole derivatives; benzimidazolones; anti-inflammatory steroids such as androstane and derivatives thereof; VLA-4 inhibitors; gama-aminobutyric acid (GABA) and derivatives thereof; as well as combinations of such NSAIDS.

Corticosteroids suitable for use as therapeutic agents in association with the stent system of the present invention include but are not limited to lower potency corticosteroids such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, and hydrocortisone-17,21-dibutyrate), atclometasone, dexamethasone, flumethasone, prednisolone, and methylprednisolone, as well as higher potency corticosteroids such as clobetasol propionate, betamethasone benzoate, betamethasone diproprionate, diflorasone diacetate, fluocinonide, mometasone fliroate, triamcinolone acetonide, and combinations thereof.

Local anesthetic agents such as phenol, benzocaine, lidocaine, prilocaine and dibucaine; topical analgesics such as glycol salicylate, methyl salicylate, 1-menthol, d,l-camphor and capsaicin; and antibiotics. Suitable antibiotic agents include, but are not limited to, antibiotics of the lincomycin family (referring to a class of antibiotic agents originally recovered from streptomyces lincolnensis), antibiotics of the tetracycline family (referring to a class of antibiotic agents originally recovered from streptomyces aureofaciens), and sulfur-based antibiotics, i.e., sulfonamides. Exemplary antibiotics of the lincomycin family include but are not limited to lincomycin itself (6,8-dideoxy-6-[[(1-methyl-4-propyl-2-pyrrolidinyl)-carbonyl]amino]-1-thio-L-threo-α-D-galactooctopyranoside), clindamycin, the 7-deoxy, 7-chloro derivative of lincomycin (i.e., 7-chloro-6,7,8-trideoxy-6-[[(1-methyl-4-propyl-2-pyrrolidinyl)carbonyl]amino]-1-thio-L-threo-α-D-galacto-octopyrano-side), and related compounds, as well as pharmacologically acceptable salts and esters thereof. Exemplary antibiotics of the tetracycline family include tetracycline itself [4-(dimethylamino)-1,4,4α,5,5α,6,11,12α-octahydro-3,6,12,12α-pentahydroxy-6-methyl-1,1′-dioxo-2-naphthacenecarboxamide], chlortetracycline, oxytetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium.

Therapeutic agents which are present on the exterior of stent 90 an also include additives such as permeability enhancers, control-release additives, and the like, including but not limited to sulfoxides such as dimethylsulfoxide (DMSO) and decylmethylsulfoxide (C₁₀MSO); ethers such as diethylene glycol monoethyl ether (available commercially as Transcutol™) and diethylene glycol monomethyl ether; surfactants such as sodium laurate, sodium lauryl sulfate, cetyltrimethylammonium bromide, benzalkonium chloride, Poloxamer (231, 182, 184), TWEEN® (20, 40, 60, 80) and lecithin; the 1-substituted azacycloheptan-2-ones, such as 1-n-dodecylcyclazacycloheptan-2-one (available under the trademark Azone™ from Nelson Research & Development Co., Irvine, Calif.).

In continued reference to FIG. 1, pusher catheter 100 is typically a polyethylene or other suitable polymeric catheter which allows for flexibility. This flexibility allows for the assembly to reach small or difficult to access vessels or cavities. As described herein, pusher catheter 100 can be of a similar diameter to the exterior diameter of the proximal flange 94 of stent 90. In use herein, pusher catheter 100 can be used to direct the endoscope-dilator cap assembly 50, and/or the stent 90, along guide wire 14 towards a stricture or other desired location needing dilation within a mammalian body.

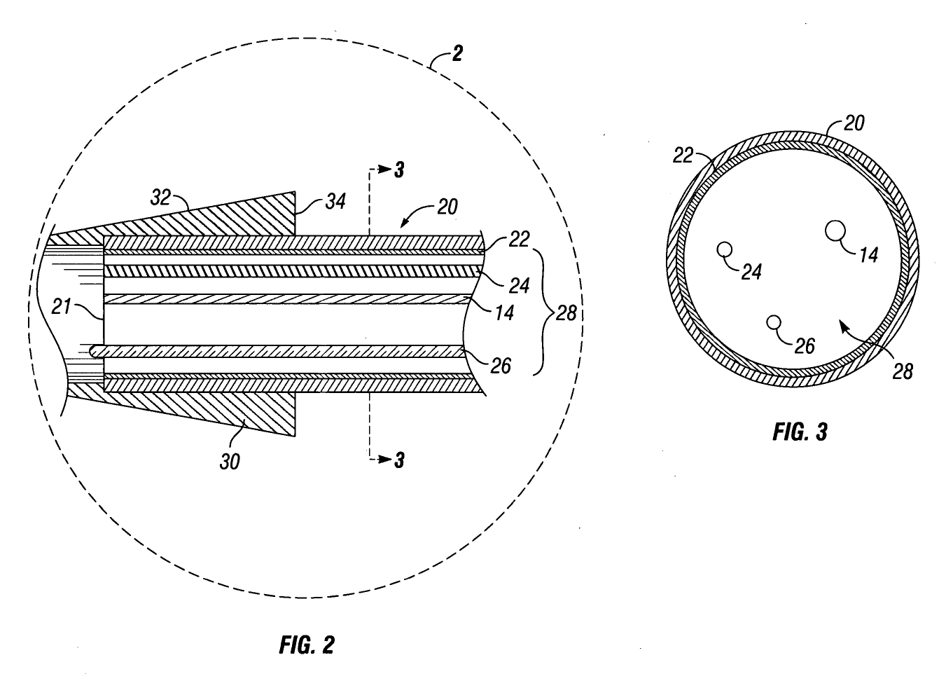

Turning now to FIG. 2, an enlargement of portion “A” of FIG. 1 is illustrated. As shown therein, endoscope 20, which can be any endoscope known in the art, typically comprises a lens 26 and a plurality, or bundle, of optic fibers 24, which can be encased in a sheath 22 to protect their integrity and the rest of the image fiber bundle 28. The optic fibers 24 are typically exposed at the distal end 21 of the endoscope so as to transmit light from a light connector (not shown) beyond the distal tip of the endoscope to illuminate the object of interest. The endoscope typically can optionally further comprise an optic junction connecting the bundle to an image connector, both of which are well known in the art.

FIG. 3 illustrates a cross-section of a portion of the endoscope-dilator cap assembly of FIG. 2, taken along line B-B. As shown in FIG. 3, the sheath 22 of the endoscope 20 encloses an image fiber bundle 28 comprising the guide wire 14 and one or more optic fibers 24 for transmitting light to be received by an optic lens 26 and focused on the end of the bundle back to the image connector for transmission to an appropriate focusing coupler, remote head, camera processor, video tape recorder and/or monitor, as necessary. The optic lens 26 can be a glass gradient index lens coupled to the distal end 21 of the endoscope's optic fiber bundle by any of a number of known, suitable adhesives, such as an acrylic ester ultraviolet cured adhesive.

Turning now to FIGS. 4A-4F, methods of use of the assemblies described above are illustrated. As illustrated in FIG. 4A, deployment of assembly in accordance with the present disclosure within a vessel or cavity 12, such as the esophagus, is considered appropriate when a tumor, lesion, or other stricture 15 has constricted the vessel or cavity interior passageway to a diameter several millimeters less than the norm. For example, in the case of esophageal strictures in humans, a constriction to a diameter of less than about 15 mm represents a severe constriction in light of the normal passageway diameter of about 22 mm. As shown in FIG. 4A, deployment begins by the oral insertion and position of guide wire 14 through and past stricture 15 in the esophageal wall 12, using an endoscope 20 in a manner well known in connection with treating not only vessels and cavities such as the esophagus, but other body lumens, such as arteries. In accordance with one aspect of the disclosure, guide wire 14 is inserted to a point such that the distal end 17 of the guide wire is at least several millimeters beyond the end of the stricture 15. As described previously, the guide wire 14 can have a diameter ranging from about 0.5 mm to about 2 mm. Once the guide wire has been properly positioned, the endoscope is withdrawn.

FIG. 4B illustrates the next step in the general method of dilating and delivering a stent into a body cavity. With the guide wire 14 in place, deployment of the endoscope-dilator assembly 50 is initiated. More particularly, as illustrated in FIG. 4B, customizable endoscope-dilator assembly 50 is assembled as described previously, and is then directed distally along guide wire 14 towards the stricture 15. As the distal end of assembly 50 encounters the proximal end of the stricture, the assembly is carefully moved distally and introduced into the stricture. The tapering of the distal end of the dilator cap 30 towards the end 36 allows the endoscope-dilator assembly to more easily follow guide wire 14 through the stricture 15 before entering the cavity space beyond the stricture. The tapered edge 32 of the dilator cap 30 further functions to minimize the chance of accidental perforation of the delivery assembly during its introduction into the vessel or cavity. Throughout this step, the assembly is guided by endoscope 20 attached to the dilator cap.

As pertains to the endoscope-dilator assembly 50 of FIG. 4B (as well as the other associated figures of this disclosure), while it is illustrated that dilator cap 30 extends only partially the axial length of the stricture 15, this is by no means meant to be limiting. Optionally, the length of the dilator cap 30 can be selected so as to be substantially the same length or longer as the stricture 15 which it is acting to dilate prior to stent insertion. Its length will depend upon the individual circumstance and the nature and recoil rate of the stricture itself following dilation. In general, however, the length of the dilator cap 30 coupled to endoscope 20 is not more than one quarter of the length of the endoscope.

In related aspects of the present disclosure, the medical operator can chose to simply dilate the stricture, in which case the endoscope-dilator assembly 50 is removed with the guide wire and the patient is released. In other aspects, the medical operator can choose to continue the dilation procedure with different sizes of dilator caps 30. In this instance, the operator can retract the endoscope-dilator assembly, leaving the guide wire in position, exchange the dilator cap in the assembly for another dilator cap, such as found in a dilator cap kit, and reinsert the endoscope-dilator assembly down the guide wire. If the procedure includes insertion of a stent, the endoscope-dilator assembly can be removed and a stent 90 and a pusher catheter 100 included with the assembly. Alternatively, the stent and pusher catheter can be preassembled with the endoscope-dilator assembly so that removal of the dilator cap prior to placement of the stent is unnecessary. The stent 90 is inserted over guide wire 14 and the proximal end of endoscope 20, and is thus directed toward the desired treatment location as it is moved distally. Distal movement of the stent is by use of a pusher catheter 100.

Now referencing FIG. 4C, distal flange 96 of the stent 90 eventually encounters the stricture 15, which has been initially dilated by endoscope-dilator assembly 50. At this point, the stent 90 is firmly but carefully moved further in the distal direction, using the tapered, flared edge 99 of the distal flange 96 of stent 90 to dilate the region of the stricture as it is moved. Once the distal flange 96 of the stent has entered the stricture 15, medial region 92 and distal flange 96 are used to further dilate the stricture as the stent 90 continues to be guided through by means of pusher catheter 100. Eventually, stent 90 will reach the point where the distal region of the stent will extend through the region of the stricture, as illustrated in FIG. 4D. Thus, both the stent 90 and the endoscope-dilator assembly 50 can dilate the region of the stricture with no need for a separate dilating tool.

FIG. 4E illustrates the withdrawal of pusher catheter 100 and the endoscope-dilator assembly 50. Withdrawal of the pusher tool 100 in the direction of the arrow is followed by withdrawal of the endoscope-dilator assembly 50, where assembly 50 is pulled proximally back and through the central core region of stent 90 until it clears stricture, 15 and the proximal flange 94 of the stent. Guide wire 14 is then withdrawn past stent 90, leaving stent 90 in place wherein medial region 92 presses against stricture 15 to maintain the esophageal passageway along the stricture. Proximal and distal flanges 94 and 96 having flared edges 98 and 99 work to fix the stent axially within the stricture, allowing for enhanced accuracy in the positioning of the stent and an enhanced ability to recover or retract the stent if necessary.

Referring now to FIG. 4F, prostheses or stents 90 suitable for use herein can optionally comprise apertures or rings 95 on or circumferentially around proximal flange 94. It is known that prostheses or stents 90 can migrate up to 20% from their point of original placement over time. The apertures or rings 95 can be included so as to provide a means by which to anchor the proximal end of the stent to the vessel wall, using an appropriate attachment means. For example, an operator can use standard surgical thread as an attachment means 93 to anchor the proximal flange 94 of the stent 90 to the interior of the esophageal wall, using apertures or rings 95. This is illustrated in FIG. 4F, wherein the finally-placed stent 90 is illustrated to be attached by way of attachment means 93, illustrated as surgical sutures, and apertures 95 in the proximal end of the stent. Apertures or rings 95 can be of any suitable shape and diameter, including circular, rectangular, and numerous other polymeric shapes. Similarly, in the instance that 95 are rings, they can be of any appropriate size, and made of appropriate material, including surgical steel, hard plastic, and metal alloys, any of which can be coated for extended use as necessary. Attachment means 93 can be any suitable material or assembly for attachment, including surgical sutures and surgical staples. Additionally, the attachment of the prosthesis or stent 90 can optionally include the use of a suitable surgical adhesive in combination with the combination of apertures or rings 95 and attachment means 93.

Suitable sutures for use in accordance with aspects of the present invention include those made of natural materials (e.g., silk) or polymeric materials (e.g., poly(ethyeleneimine), nylon, polyester, and the like), or variations thereof (such as VICRYL™ Polyglactin 910 surgical suture, from Ethicon, Inc. Somerville, N.J.) and have a strength, diameter, feel, and tie ability suitable for the presently described use. The suture can be uncoated, or coated with any number of suitable coatings, including wax (beeswax, petroleum wax, polyethylene wax, or others), silicone (Dow Corning silicone fluid 202A or others), silicone rubbers (Nusil Med 2245, Nusil Med 2174 with a bonding catalyst, or others) PTFE (Teflon®, Hostaflon, or others), PBA (polybutylate acid), ethyl cellulose (Filodel™) or other coatings, to improve lubricity of the braid, knot security, or abrasion resistance, for example. Other suitable coatings include antibacterial agents and/or dyes.

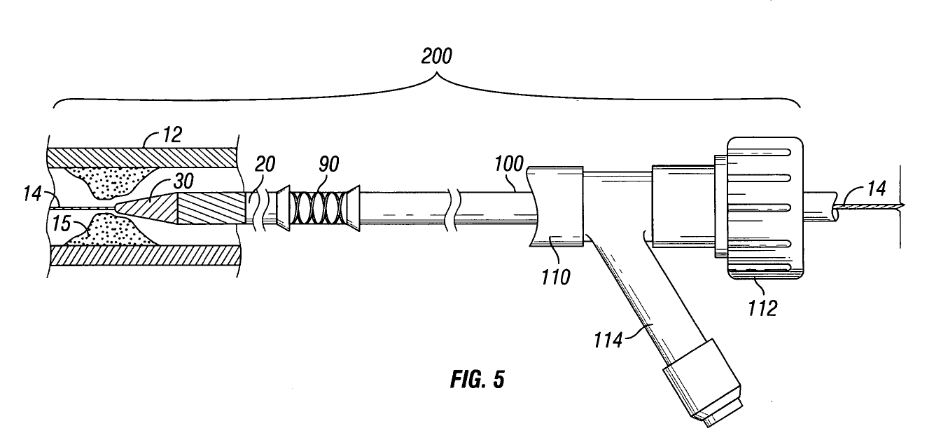

FIG. 5 illustrates a fragmentary and partial side sectional view of a complete assembly as it is inserted into a vessel. A longitudinal side section of the assembly of the present disclosure is shown in FIG. 5. The proximal end of the assembly 10 of the present invention can include a Y-connector 110 and an optional rotating adaptor, both of which are elements known to those of skill in the art. A Y-connector cap 112 acts to close off the proximal end of the Y-connector and compresses a sealing element (not shown) in the barrel of the Y-connector. The Y-connector includes an irrigation duct 114 for injecting fluid through the passage containing the endoscope-dilator assembly, and ultimately into the primary lumen of the assembly.

FIG. 6A illustrates a detailed view of an endoscope-dilator assembly. A detail of the front end of a dilator-endoscope assembly 50 in accordance with aspects of the present invention is shown, wherein endoscope 20 is inserted into the proximal end 34 of dilator cap 30, forming a tight attachment, or coupling. This coupling can be further strengthened by the inclusion or use of adhesives, or threadable connections (not shown).

In at least one embodiment, endoscope 20 has a first diameter d₁, which is just slightly less than the diameter d₂ of the open, proximal end of dilator cap 30, allowing distal end 21 of endoscope 20 to be inserted an axial length L₁ into the open, proximal end of dilator cap 30, whereupon it stops due to having a diameter d₁ substantially the same as the inner diameter d₄ of dilator cap 30 at that point. As also illustrated in FIG. 6A, dilator cap 30 has a tapered edge 32, such that the diameter d₃ of the opening at the distal end 36 is less than the diameter d₂ of the proximal end of the dilator.

FIG. 6B illustrates an alternate embodiment of the endoscope-dilator assembly shown in FIG. 6A. As shown therein, dilator cap 30 can have an inner bore 23 with a ledge 25, suitable for use in coupling endoscope 20 with cap 30 to form endoscope-dilator assembly 50. In accordance with this aspect of the disclosure, endoscope 20, having a first diameter d, as described in relation to FIG. 6A that is less than the diameter of the open, proximal end 34 of dilator cap 30, can be slidably inserted axially into the open end 34 of the dilator cap. Endoscope 20 is pushed axially until the distal end 21 comes to rest against ledge 25 extending from the inner bore 23 of dilator cap 30. Ledge 25 thereby provides a means of retaining endoscope 20 within dilator cap 30 following this coupling.

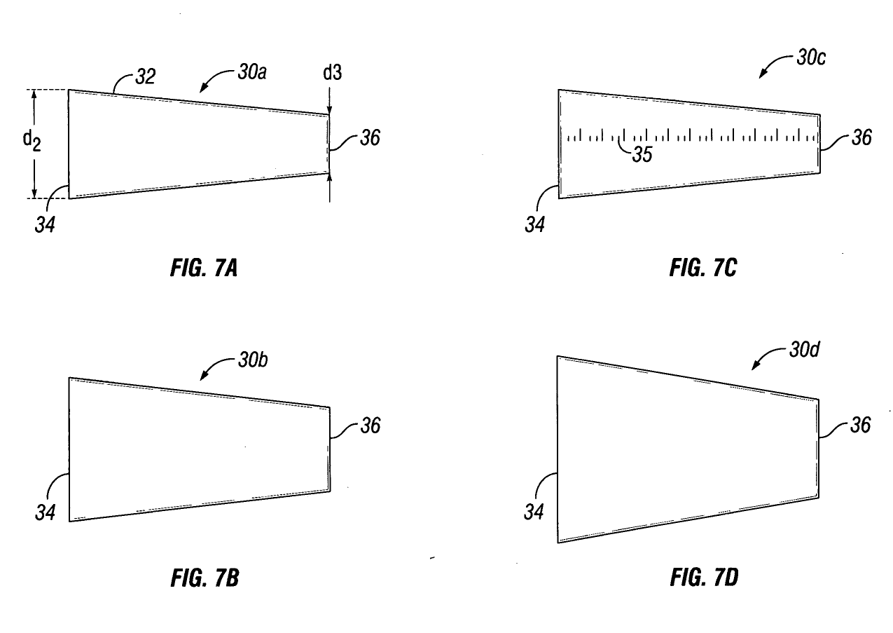

Turning now to FIG. 7, a dilator cap kit comprising a plurality of dilator caps adapted to be used with the delivery systems assemblies and methods of the present disclosure is illustrated. As shown therein, a plurality of different sized dilator caps 30a, 30b, 30c and 30d is shown. However, one will realize that such dilator cap kits can contain any number of differently-sized dilator caps, and would not be limited to the four exemplary dilator caps illustrated. Referring now to dilator cap 30a, but recognizing that the same features can be applied to each of the dilator caps 30a-30d illustrated within FIG. 7, each of the dilator caps comprise a proximal end 34, a distal end 36, and are generally conical in shape having a tapered edge 32 between the proximal and distal ends. Consequently, the diameter of proximal end d₂ will be greater than the diameter d₃ of distal end 34. Typically, the diameter of the proximal end of dilators 30a-30d can range from about 5 mm to about 15 mm, including about 6 mm, about 7 mm, about 8 mm, about 9 mm, about 10 mm, about 11 mm, about 12 mm, about 13 mm, and about 14 mm, as well as diameters between any two of these values, such as about 13.5 mm. Similarly, the typical diameter of the distal end 34 of dilators 30a, 30b, 30c and 30d in the kit of FIG. 7 can range from about 2 mm to about 8 mm, and advantageously from about 3 mm to about 6 mm in diameter, including about 3 mm, about 4 mm, about 5 mm, as well as diameters between any two of these values, such as from about 2 mm to about 5 mm. For example and without limitation, dilator cap 30a can have a proximal end diameter d₂ of about 5 mm and a distal end diameter d₃ of about 3 mm; dilator cap 30b can have a proximal end diameter d₂ of about 6 mm and a distal end diameter d₃ of about 4 mm; dilator cap 30c can have a proximal end diameter d₂ of about 5 mm and a distal end diameter d₃ of about 2 mm; and, dilator cap 30d can have a proximal end diameter d₂ of about 9 mm and a distal end diameter d₃ of about 5 mm. As further optionally illustrated therein, the transparent or translucent dilators can have one or more graduations 35 engraved or printed within or onto the exterior of the cap itself so as to allow the operator to distinguish length or other suitable parameters.

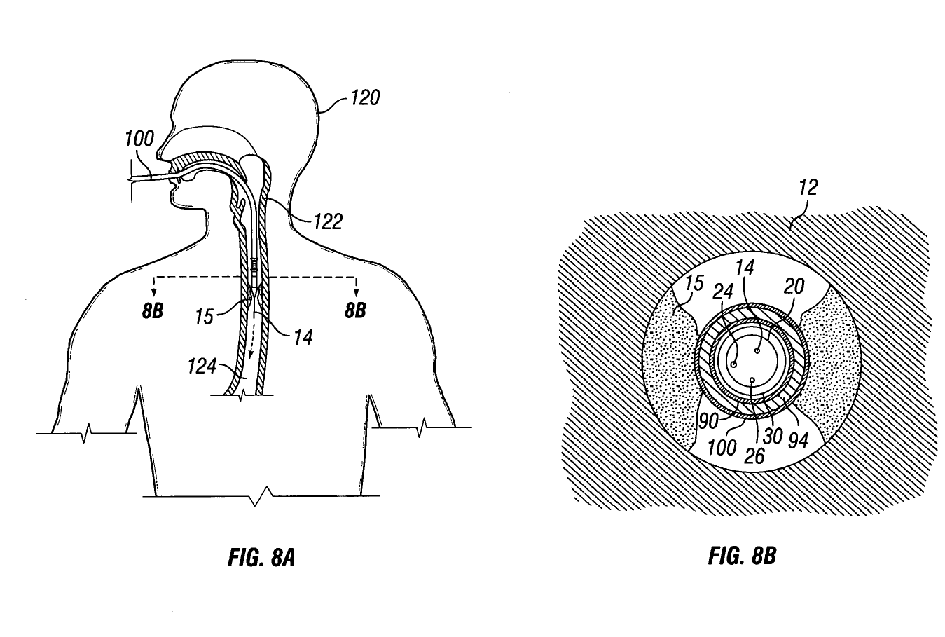

FIGS. 8A and 8B illustrate a cut-away view of an assembly in accordance with the present disclosure during placement in a human patient, and a top view of the assembly of FIG. 8A, taken along line C-C of FIG. 8A, respectively. As shown in FIG. 8A, in a typical application of the assemblies described herein, a human patient 120 having a stricture 15 lining the interior tracheal wall 122 of their esophagus 124, has an assembly in accordance with the present disclosure inserted within their esophagus. As is clearly seen, the assembly is pushed down the esophagus towards the stricture 15, in the direction of the arrow, using pusher catheter 100. In the cross-sectional view of FIG. 8B, taken along line C-C of FIG. 8A, the view is looking downward from the proximal end of pusher 100 towards the distal end of guide wire 14, respectively. As can be seen, the assemblies described herein have a generally concentric relationship to each other. More specifically, at the inner-most portion of the assembly is the endoscope 20, having guide wire 14, optic fiber(s) 24, and optical lens 26 passing there through. Coupled to the distal end of the endoscope is the dilator cap 30, forming an endoscope-dilator assembly. Stent 90 is slidably inserted along endoscope 20, and is moved forward towards the endoscope-dilator assembly by pusher catheter 100, which pushes stent 90 along by contacting flange 94 of stent 90. As is evident from FIG. 8B, the endoscope-dilator assembly can be removed in a single operation while stent 90 is retained within the body to act outwardly against stricture 15.

While compositions and methods are described in terms of “comprising” various components or steps (interpreted as meaning “including, but not limited to”), the compositions and methods can also “consist essentially of” or “consist of” the various components and steps, such terminology should be interpreted as defining essentially closed-member groups.

The term “mammal”, as used herein, refers to any of the various warm-blooded vertebrate animals of the class Mammalia which can be treated using the assemblies and methods disclosed herein, including but not limited to humans, canines, equines, felines, bovines, and the like.

Additionally, the term “coupled,” “coupling,” and like terms are used broadly herein and can include any method or device for securing, binding, bonding, fastening, attaching, joining, inserting therein, forming thereon or therein, communicating, or otherwise associating, for example, mechanically, magnetically, electrically, chemically, directly or indirectly with intermediate elements, one or more pieces of members together and can further include integrally forming one functional member with another. The coupling can occur in any direction, including rotationally.

The invention has been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicant, but rather, in conformity with the patent laws, Applicant intends to protect all such modifications and improvements to the full extent that such falls within the scope or range of equivalent of the following claims.

What is claimed is:

1. An assembly for the delivery of a prosthesis in a body vessel or cavity of a mammal, the assembly comprising:
a guide wire;
an endoscope having a distal end and a proximal end;
a dilator cap having a distal end, a proximal end, and a tapered shape wherein the proximal end has a diameter greater than the diameter of the distal end; and
wherein the proximal end of the dilator cap is coupled to the distal end of the endoscope, and
wherein the dilator cap is transparent or translucent.

2. The assembly of claim 1, wherein the dilator cap is selected from a kit comprising a plurality of dilator caps, the plurality of dilator caps each having different proximal end diameters.

3. The assembly claim 1 further comprising a flexible stent delivery device and a stent removably coupled to the device.

4. A method for delivering a prosthesis into a body vessel or cavity of a mammal using the assembly of claim 1, the method comprising:
a) inserting a guide wire within a body vessel or cavity;
b) coupling a first dilator cap to the distal end of an endoscope to form a endoscope-dilator assembly;
c) extending the endoscope-dilator assembly through the interior of the vessel or cavity to a predetermined point so as to effectively dilate the predetermined point; and
d) withdrawing the endoscope-dilator assembly.

5. The method of claim 4, further comprising:
selecting a second dilator cap from a kit comprising a plurality of dilator caps, the second dilator cap having proximal end diameter different from the proximal end diameter of the first dilator cap;
coupling the second dilator cap to the distal end of the endoscope to form a second endoscope-dilator assembly; and
extending the second endoscope-dilator assembly through the interior of the vessel or cavity to the predetermined point.

6. An assembly for the delivery and fixation of a stent in a body vessel or cavity, the assembly comprising:
a guide wire;
an endoscope having a distal end and a proximal end;
a dilator cap having a distal end, a proximal end, and a length no more than one quarter of the length of the endoscope;
a stent; and
a flexible stent delivery device,
the dilator cap being transparent or translucent and coupled to the distal end of the endoscope.

7. The assembly of claim 6, wherein the dilator cap is selected from a kit comprising a plurality of dilator caps, the plurality of dilator caps each having different proximal end and/or distal end diameters.

8. The assembly of claim 6, wherein the stent comprises one or more apertures or rings circumferentially located on a flange of the stent.

9. A method for delivering a stent into a body vessel or cavity, the method comprising:
a) providing a stent delivery device;
b) inserting a guide wire having a distal tip and a proximal end within a body vessel or cavity until the guide wire distal tip reaches a desired location in the body vessel or cavity;
c) extending an endoscope having a dilator attached to the distal end thereof through the interior of the vessel or cavity to a predetermined point;
d) inserting a stent through the vessel or cavity to the predetermined point with a stent delivery device, the stent being slidably disposed on the endoscope;
e) withdrawing the endoscope with attached dilator and the guide wire from the body vessel or cavity; and
f) withdrawing the stent delivery device from the body vessel or cavity, wherein the method of delivering is performed independent of a stent expanding device.

10. The method of claim 9, further comprising attaching the stent to a wall of the vessel or cavity using an attachment means that is capable of passing through one or more apertures or rings in a proximal flange of the stent and into a wall of the vessel or cavity.

11. The method of claim 10, wherein the attachment means comprises a surgical suture.

12. The method of claim 9, wherein the endoscope-dilator assembly, guide wire, and stent delivery device are withdrawn simultaneously.

13. A method for delivering a stent into the esophagus of a mammal for treating esophageal strictures, the method comprising:
a) extending a guide wire into the esophagus and extending the guide wire past a stricture in the esophagus;
b) inserting an endoscope having a dilator affixed thereto into the esophagus, extending through the esophageal stricture;
c) inserting a stent into the esophagus, over the endoscope;
d) inserting the stent into the esophageal stricture using a pusher catheter; and
e) withdrawing the endoscope having the dilator affixed thereto from the stricture.

14. The method of claim 13, further comprising withdrawing the pusher catheter and the guide wire.

15. The method of claim 13, further comprising withdrawing the pusher catheter, guide wire, and endoscope having the dilator affixed thereto simultaneously.

16. The method of claim 13, further comprising attaching the stent to a wall of the vessel or cavity using an attachment means that is capable of passing through one or more apertures or rings in a proximal flange of the stent and into a wall of the vessel or cavity.

17. A kit for use in endoscopic surgery, the kit comprising:
a plurality of conically-shaped dilator caps having a distal end, a proximal end, and a tapered edge between the distal end and the proximal end,
wherein the dilators:
are translucent or transparent;
have a proximal end with a diameter ranging from about 5 mm to about 15 mm; and
have a distal end with a diameter ranging from about 2 mm to about 8 mm.

18. The kit of claim 17, wherein the dilators further comprise at least-one radio-opaque element.

19. The kit of claim 17, wherein at least one of the dilators has graduations.

20. A stent, comprising:
a distal flange;
a proximal flange;
a medial sleeve intermediate between the distal flange and the proximal flange; and
a support structure within the medial sleeve,
wherein the proximal flange comprises one or more attachment means for the attachment of the stent to a biological body.
